(19) United States
(12) Patent Application Publication
(10) Pub. No.: US 2013/0090355 A1
(43) Pub. Date: Apr. 11, 2013

(54) CHEMICAL AGENTS FOR THE PREVENTION OF INHIBITION OR TUMOR METASTASIS

(71) Applicant: Anne R. Bresnick, Pelham, NY (US)

(72) Inventor: Anne R. Bresnick, Pelham, NY (US)

(73) Assignee: ALBERT EINSTEIN COLLEGE OF MEDICINE OF YESHIVA UNIVERSITY, Bronx, NY (US)

(21) Appl. No.: 13/640,596

(22) PCT Filed: Apr. 28, 2011
(86) PCT No.: PCT/US11/00740

§ 371 Date: Nov. 7, 2012

Related U.S. Application Data
(60) Provisional application No. 61/396,062, filed on May 21, 2010.

(51) Int. Cl. A61K 31/4709; A61K 31/382; A61K 31/122; A61K 31/16; A61K 31/136; A61K 31/47; A61K 31/235

(52) U.S. Cl. CPC A61K 31/4709; A61K 31/47; A61K 31/382; A61K 31/235; A61K 31/16; A61K 31/136; A61K 31/122
USPC 514/314; 514/311; 514/434; 514/544; 514/630; 514/656; 514/676; 514/680; 514/681

(57) ABSTRACT

The present invention provides methods of preventing or inhibiting tumor metastasis in a subject by administering a therapeutically effective amount of (1) a compound from a group of enumerated compounds, or a pharmaceutically acceptable salt thereof; (2) an agent that covalently modifies at least one cysteine residue of S100A4 protein; or (3) an agent that inhibits the interaction between S100A4 and myosin-IIA.

### CROSS-REFERENCE TO RELATED APPLICATION

This application claims the benefit of U.S. Provisional Patent Application No. 61/396,062, filed May 21, 2010, the content of which is hereby incorporated by reference into the subject application.

### STATEMENT OF GOVERNMENT SUPPORT

This invention was made with government support under grant number CA129598 awarded by the National Cancer Institute, National Institutes of Health, U.S. Department of Health and Human Services. The government has certain rights in the invention.

### FIELD OF THE INVENTION

The present invention relates generally to chemical agents for the prevention or inhibition of tumor metastasis.

### BACKGROUND OF THE INVENTION

Throughout this application various publications are referred to in parenthesis. Full citations for these references may be found at the end of the specification. The disclosures of these publications are hereby incorporated by reference in their entirety into the subject application to more fully describe the art to which the subject invention pertains.

The leading cause of mortality in cancer patients is the consequence of malignant cells leaving the primary tumor, traveling to distant sites within the body and forming secondary tumors (metastasis)(1). From a clinical standpoint, the prevention or inhibition of metastasis is vital to the treatment of cancer. Since metastasis impacts many types of cancer (e.g. breast, prostate, etc.), this unmet clinical need represents an opportunity to positively impact the health of large patient populations. The transition from benign tumor growth to malignancy is manifested by the ability of the tumor cell to traverse tissue barriers and invade surrounding tissues.

S100A4 is overexpressed in a number of cancers including breast and prostate, and has been demonstrated by gain and loss of function studies to play a direct role in tumor metastasis. It has been shown that S100A4 modulates cell motility through its interaction with myosin-IIA.

Most current cancer therapeutics block tumor cell proliferation. However, the success of those treatments is limited by metastatic disease. As a consequence, there is a recognized need for cancer therapeutics that directly target metastatic disease.

The present invention addresses this need and provides a novel platform technology for the treatment of metastatic disease for a number of cancers by identifying several inhibitors that prevent cancer cell migration.

### SUMMARY OF THE INVENTION

The present invention provides a method for preventing or inhibiting tumor metastasis in a subject, the method comprising administering a therapeutically effective amount of a compound selected from the group consisting of

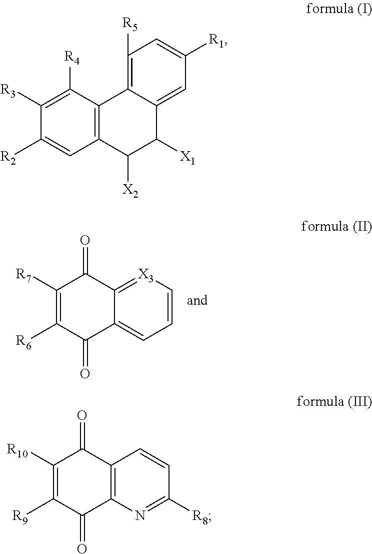

wherein

X1 is ( )═O or ( )—OH;

X2 is selected from the group consisting of

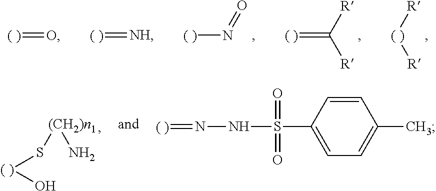

wherein R′ is a halogen and n₁ is an integer 0-3;

X3 is CH or N;

R1 is selected from the group consisting of H, Br, Cl, F, I, At, and

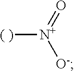

R2, R3 and R4 are independently selected from the group consisting of

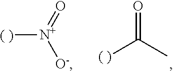

### H, NH₂, Cl, Br, F, I, and At;

R5 is H or

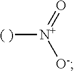

R6 is selected from the group consisting of Cl, F, Br, I, At, NH₂, H,

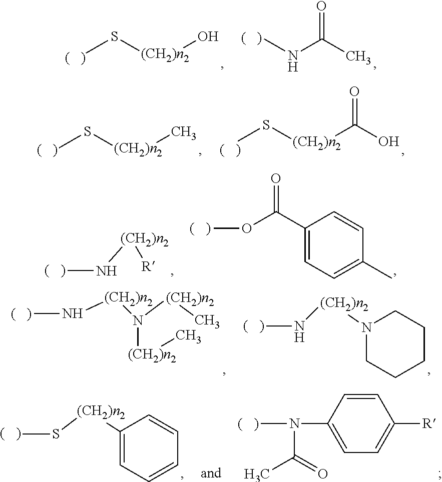

wherein R′ is a halogen and each n₂ is independently an integer 0-5;

R7 is selected from the group consisting of H, Cl, Br, F, I, At, CH₃,

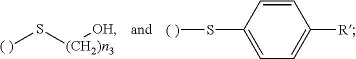

wherein R′ is a halogen and n₃ is an integer 0-5;

R8 is CH₃ or H;

R9 is selected from the group consisting of H, NH₂,

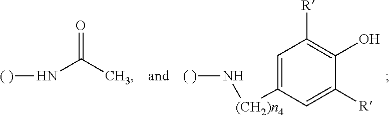

wherein R′ is a halogen and n₄ is an integer 0-5; and

R10 is H or

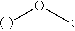

wherein R′ is a halogen, n₁ is an integer 0-3, and n₂ is an integer 0-5;

wherein ( ) is the point of attachment of the X or R group to the ring structure;

or a pharmaceutically acceptable salt thereof.

The present invention also provides a method of inhibiting tumor metastasis in a subject, the method comprising administering a therapeutically effective amount of a compound selected from the group consisting of:

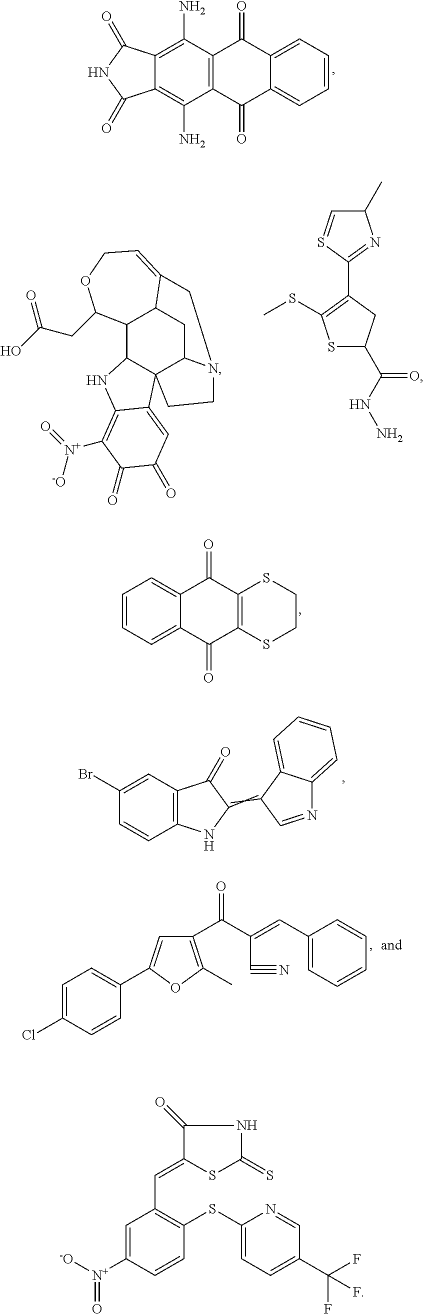

The present invention also provides a method of preventing or inhibiting tumor metastasis in a subject, the method comprising administering to the subject a therapeutically effective amount of an agent that covalently modifies at least one cysteine residue of an S100A4 protein, wherein the modification of at least one cysteine residue of S100A4 protein prevents or inhibits tumor metastasis in the subject.

The present invention further provides a method of preventing or inhibiting tumor metastasis in a subject, the method comprising administering to the subject a therapeutically effective amount of an agent that inhibits the interaction between S100A4 and myosin-IIA, wherein inhibition of the interaction between S100A4 and myosin-IIA prevents or inhibits tumor metastasis in the subject.

The present invention provides the compound or agent as described in any of the method claims for use in preventing or inhibiting tumor metastasis in a subject.

### BRIEF DESCRIPTION OF THE DRAWINGS

FIG. 1. Ribbon diagram of apo and Ca²⁺-S100A4.

FIG. 2A-2B. (A) Cartoon of FITC-MIIA¹⁹⁰⁸⁻¹⁹²³ binding to S100A4. (B) Fluorescence anisotropy measurements of S100A4 binding to FITC-MIIA¹⁹⁰⁸⁻¹⁹²³. Values represent the mean±sd from three independent experiments.

FIG. 3A-3D. Mass spectrum of wild-type S100A4 (A) and following treatment with NSC 95397 (B). Mass spectrum of C81S/C86S S100A4 (C) and following treatment with NSC 95397 (D).

FIG. 4. Boyden chamber assay examining the effects of lead compounds on MDA-MB-231 chemotaxis. Cells were serum starved for 5 hrs and plated into the upper chamber of a transwell in serum-free medium. Complete medium (5% FBS) was added to the lower chamber and the cells were allowed to migrate for 24 hr. Cells that penetrated the filter were stained with DAPI and quantified by fluorescence microscopy using 10 fields per filter. MDA-MB-231 cells showed a 23-fold increase in chemotaxis in response to serum.

### DETAILED DESCRIPTION OF THE INVENTION

The present invention provides a method of preventing or inhibiting tumor metastasis in a subject, the method comprising administering a therapeutically effective amount of a compound selected from the group consisting of

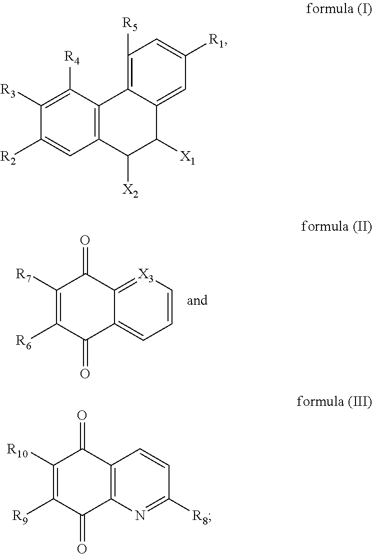

wherein

X1 is ( )═O or ( )—OH;

X2 is selected from the group consisting of

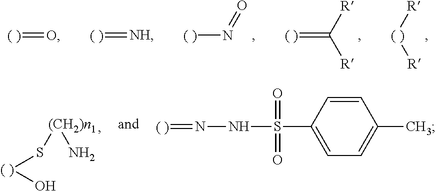

wherein R′ is a halogen and n₁ is an integer 0-3;

X3 is CH or N;

R1 is selected from the group consisting of H, Br, Cl, F, I, At, and

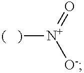

R2, R3 and R4 are independently selected from the group consisting of

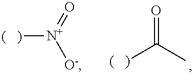

### H, NH₂, Cl, Br, F, I, and At;

R5 is H or

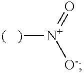

R6 is selected from the group consisting of Cl, F, Br, I, At, NH₂, H,

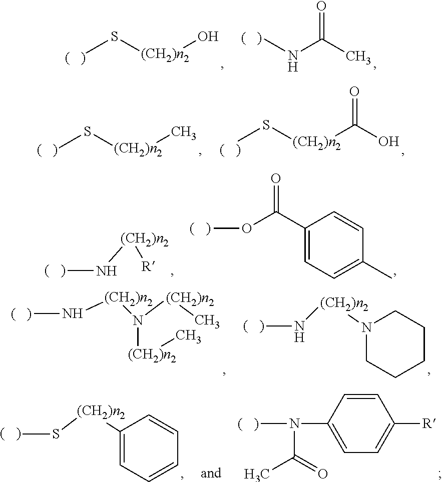

wherein R′ is a halogen and each n₂ is independently an integer 0-5;

R7 is selected from the group consisting of H, Cl, Br, F, I, At, CH₃,

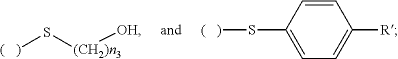

wherein R′ is a halogen and n₃ is an integer 0-5;

R8 is CH₃ or H;

R9 is selected from the group consisting of H, NH₂,

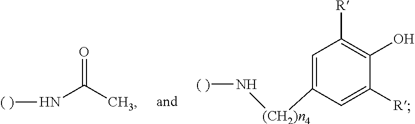

wherein R′ is a halogen and n₄ is an integer 0-5; and

R10 is H or

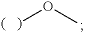

wherein R′ is a halogen, n₁ is an integer 0-3, and n₂ is an integer 0-5;

wherein ( ) is the point of attachment of the X or R group to the ring structure;

or a pharmaceutically acceptable salt thereof.

The present invention also provides a method of inhibiting tumor metastasis in a subject, the method comprising administering a therapeutically effective amount of a compound selected from the group consisting of:

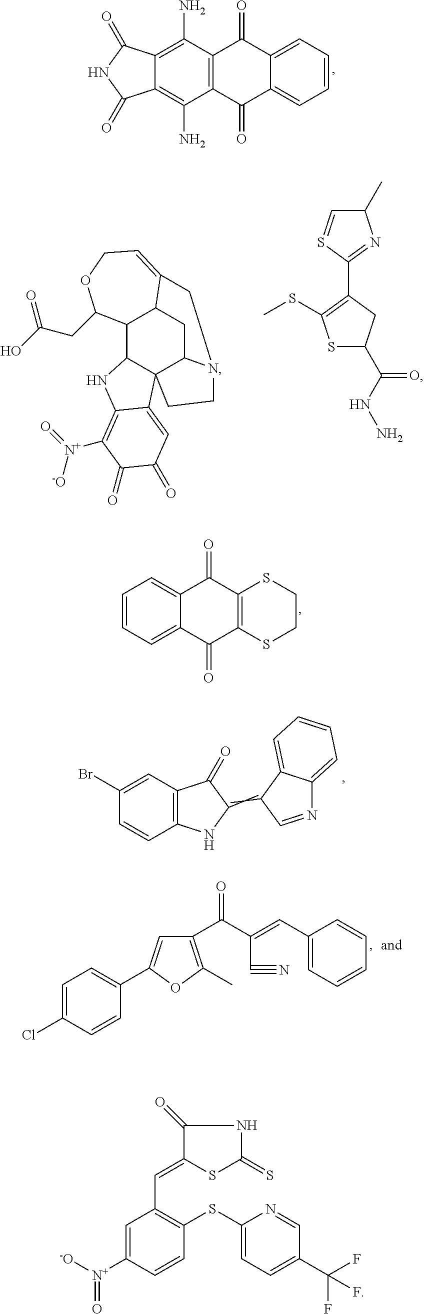

The compound may be an optical isomer and stereoisomer of the structures disclosed herein.

A salt of the compound may include a salt derived from inorganic or organic acids, including, for example, an acid salt such as acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2 naphthalenesulfonate, nicotinate, nitrate, oxalate, palmoate, pectinate, persulfate, 3 phenylpropionate, phosphate, picrate, pivalate, propionate, p-toluenesulfonate, salicylate, succinate, sulfate, tartrate, thiocyanate, or undecanoate.

The compound may be any compound of formula (I). For example, the compound may be a compound of formula (I) selected from the group consisting of

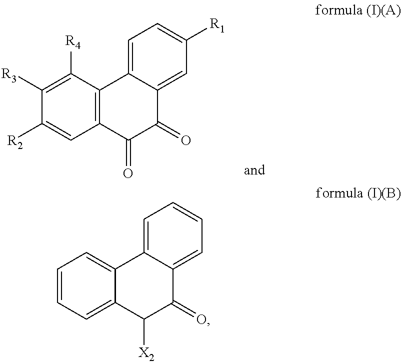

wherein

X2 is selected from the group consisting of

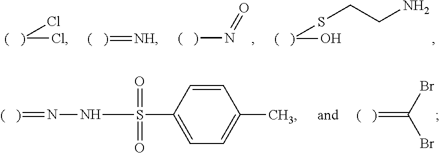

R1 is selected from the group consisting of H, Br, Cl, and

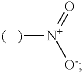

R2 is selected from the group consisting of H, NH₂, Cl, Br and

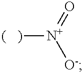

R3 is H or

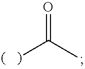

and

R4 is H or

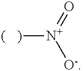

The compound may be a compound of formula (II) selected from the group consisting of

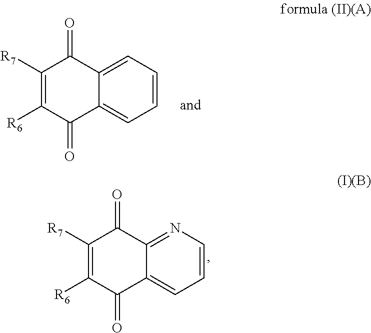

wherein

R6 is selected from the group consisting of Cl, F, Br, H,

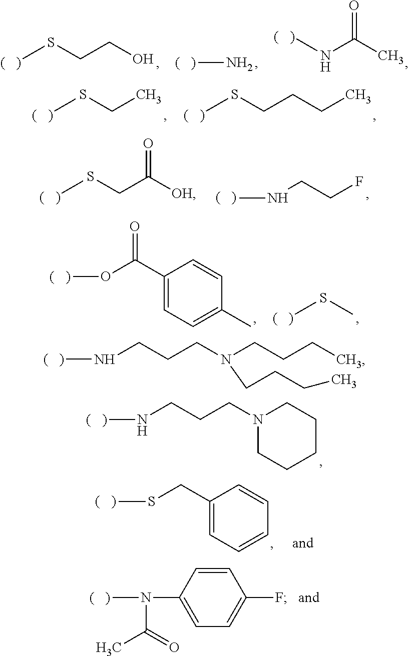

R7 is selected from the group consisting of Cl, Br, H, CH₃,

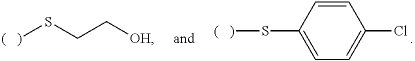

The compound may be any compound of formula (III). For example, the compound may be a compound of formula (III), wherein

R9 is selected from the group consisting of H, NH₂,

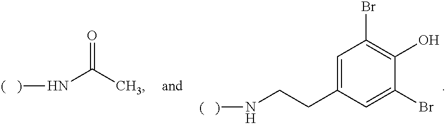

For example, the compound may be 2,3-dihydrobenzo[g][1,4]benzodithiine-5,10-dione, 2,3-bis(2-hydroxyethylsulfanyl)naphthalene-1,4-dione, 2-(2-hydroxyethylsulfanyl)naphthalene-1,4-dione, 3-(1,4-dioxonaphthalen-2-yl)sulfanylpropanoic acid, 2-ethylsulfanylnaphthalene-1,4-dione, 4,11-diaminonaphtho[2,3-f]isoindole-1,3,5,10-tetrone, 2-(3-methyl-1,4-dioxonaphthalen-2-yl)sulfanylacetic acid, 2-butylsulfanylnaphthalene-1,4-dione, 2-ethylsulfanyl-3-methylnaphthalene-1,4-dione, 2-(2-hydroxyethylsulfanyl)-3-methylnaphthalene-1,4-dione, 2-methyl-3-methylsulfanylnaphthalene-1,4-dione, (1,4-dioxonaphthalen-2-yl) 4-methylbenzoate, N-[3-(4-chlorophenyl)sulfanyl-1,4-dioxonaphthalen-2-yl]acetamide, 2-benzylsulfanyl-3-methylnaphthalene-1,4-dione, N-(3-chloro-1,4-dioxonaphthalen-2-yl)-N-(4-fluorophenyl)acetamide, 2-methylquinoline-5,8-dione, N-(7-chloro-5,8-dioxoquinolin-6-yl)acetamide, 6-amino-7-chloroquinoline-5,8-dione, 7-amino-6-methoxyquinoline-5,8-dione, 6,7-dichloroquinoline-5,8-dione, quinoline-5,8-dione, 6-amino-7-bromoquinoline-5,8-dione, N-(5,8-dioxoquinolin-7-yl)acetamide, 2,3-dichloro-2,3-dihydronaphthalene-1,4-dione, 7-chloro-6-(2-fluoroethylamino)quinoline-5,8-dione, 6-aminoquinoline-5,8-dione, 2-chloro-2,3-dihydronaphthalene-1,4-dione, 6-[3-(dibutylamino)propylamino]quinoline-5,8-dione, 6-(3-piperidin-1-ylpropylamino)quinoline-5,8-dione, 2-methylsulfanylnaphthalene-1,4-dione, 2-nitrophenanthrene-9,10-dione, 2-chlorophenanthrene-9,10-dione, 2-aminophenanthrene-9,10-dione, 3-acetylphenanthrene-9,10-dione, 4-nitrophenanthrene-9,10-dione, 10,10-dichlorophenanthren-9-one, phenanthrene-9,10-dione, 10-(2-aminoethylsulfanyl)-10-hydroxyphenanthren-9-one hydrochloride, 10-iminophenanthren-9-one, 2,7-dichlorophenanthrene-9,10-dione, 2,7-dibromo-4-nitrophenanthrene-9,10-dione, 4-methyl-N—[(Z)-(10-oxophenanthren-9-ylidene)amino]benzenesulfonamide, 10-nitrosophenanthren-9-ol, 4,5-dinitrophenanthrene-9,10-dione, 2-nitro-10-nitrosophenanthren-9-ol, 2,7-dinitrophenanthrene-9,10-dione, 2,7-dibromophenanthrene-9,10-dione, 10-(dibromomethylidene)phenanthren-9-one, Cacotheline, 5-bromo-2-indol-3-ylidene-1H-indol-3-one, 7-[2-(3,5-dibromo-4-hydroxyphenyl)ethylamino]quinoline-5,8-dione, 5-methylsulfanyl-4-(4-methyl-1,3-thiazol-2-yl)thiophene-2-carbohydrazide, 2-{[5-(4-chlorophenyl)-2-methyl-3-furyl]carbonyl}-3-phenylacrylonitrile, or 5-(5-nitro-2 {[5-(trifluoromethyl)-2-pyridyl]thio}benzylidene)-2-thioxo-1,3-thiozolan-4-one.

TABLE 1
Compounds with structures and EC50 values.

| | | | EC50 |
|---|---|---|---|
| Compound | Chemical Name | Structure | (μM) |
| NSC5425/ NSC23180 | 2-nitrophenanthrene-9,10-dione 2(Nitro-9,19-phenanthrenedione) | 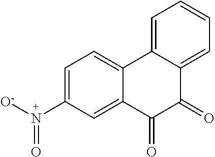 | 0.5/ 2.0 |
| NSC77642 | 2-aminophenanthrene-9,10-dione | 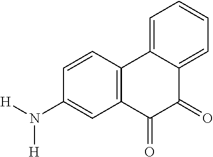 [SMILES: Nc1ccc2c(c1)C(=O)C(=O)c1ccccc1-2] | 1.6 |
| NSC102363 | 2-chlorophenanthrene-9,10-dione | 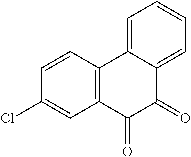 [SMILES: O=C1C(=O)c2cc(Cl)ccc2-c2ccccc21] | 1.8 |
| NSC55480 | 2,3- dihydrobenzo[g][1,4]benzodithiine- 5,10-dione (2,3-Dihydronaphtho[2,3- b][1,4]dithiine-5,10-dione) | 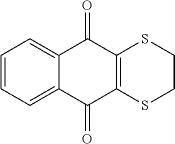 [SMILES: O=C1C2=C(SCCS2)C(=O)c2ccccc21] | 2.2 |
| NSC400689 | 3-acetylphenanthrene-9,10-dione | 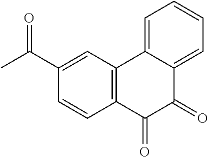 [SMILES: CC(=O)c1ccc2c(c1)-c1ccccc1C(=O)C2=O] | 2.2 |
| NSC10204 | 4-nitrophenanthrene-9,10-dione | 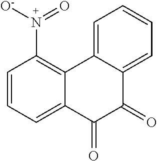 | 2.5 |
| NSC6339/ NSC135154 | 10,10-dichlorophenanthren-9-one | 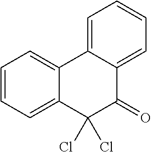 [SMILES: O=C1c2ccccc2-c2ccccc2C1(Cl)Cl] | 2.5/ 4.8 |
| NSC10446/ NSC7389 | Phenanthrene-9,10-dione | 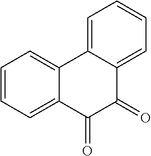 [SMILES: O=C1C(=O)c2ccccc2-c2ccccc21] | 3.8 |
| NSC139049 | 10-(2-aminoethylsulfanyl)-10- hydroxyphenanthren-9-one hydrochloride | 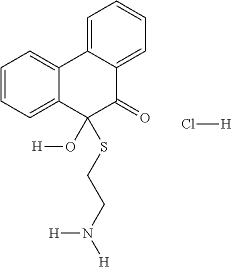 [SMILES: NCCSC1(O)C(=O)c2ccccc2-c2ccccc21 ; Cl] | 4.1/ 9.1 |
| NSC139152 | 10-iminophenanthren-9-one | 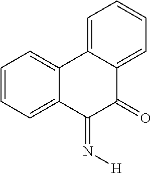 [SMILES: [H]/N=C1\C(=O)c2ccccc2-c2ccccc21] | 4.3 |
| NSC95397 | 2,3-bis(2- hydroxyethylsulfanyl)naphthalene- 1,4-dione (2,3-Bis[(2-hydroxyethyl)thio]-1,4- napthoquinone) | 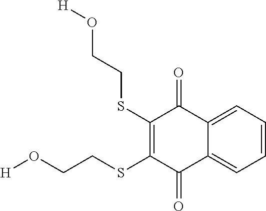 [SMILES: O=C1C(SCCO)=C(SCCO)C(=O)c2ccccc21] | 7.6 |
| NSC102381 | 2,7-dichlorophenanthrene-9,10- dione | 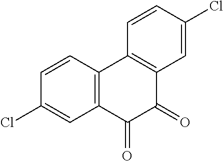 [SMILES: O=C1C(=O)c2cc(Cl)ccc2-c2ccc(Cl)cc21] | 8.1/ 8.7 |
| NSC102382 | 2,7-dibromo-4-nitrophenanthrene- 9,10-dione | 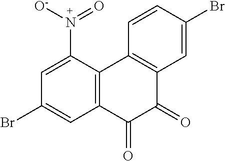 | 8.3 |
| NSC329288 | 4-methyl-N-[(Z)-(10- oxophenanthren-9- ylidene)amino]benzenesulfonamide | 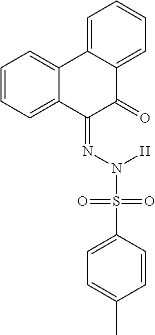 [SMILES: Cc1ccc(S(=O)(=O)N/N=C2\C(=O)c3ccccc3-c3ccccc32)cc1] | 8.4 |
| NSC682995 | 2-methylquinoline-5,8-dione | 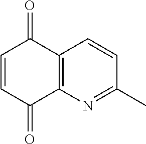 | 8.6 |
| NSC48526 | 10-nitrophenanthren-9-ol | 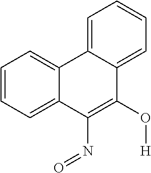 [SMILES: O=Nc1c(O)c2ccccc2c2ccccc12] | 8.6 |
| NSC102381 | 2,7-dichlorophenanthrene-9,10- dione | 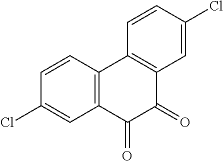 [SMILES: O=C1C(=O)c2cc(Cl)ccc2-c2ccc(Cl)cc21] | 8.7 |
| NSC84999/ NSC84990 | N-(7-chloro-5,8-dioxoquinolin-6- yl)acetamide | 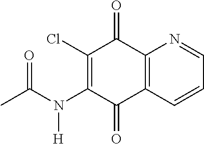 [SMILES: CC(=O)NC1=C(Cl)C(=O)c2ncccc2C1=O] | 9 |
| NSC76890 | 7-amino-6-methoxyquinoline-5,8- dione | 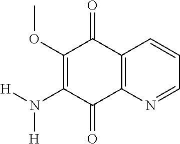 | 12 |
| NSC84998/ NSC81056 | 6-amino-7-chloroquinoline-5,8-dione | 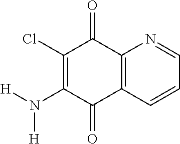 [SMILES: NC1=C(Cl)C(=O)c2ncccc2C1=O] | 13.5 |
| NSC81047 | 6,7-dichloroquinoline-5,8-dione | 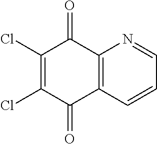 [SMILES: O=C1C(Cl)=C(Cl)C(=O)c2ncccc21] | 14 |
| NSC148596 | 2-(2- hydroxyethylsulfanyl)naphthalene- 1,4-dione | 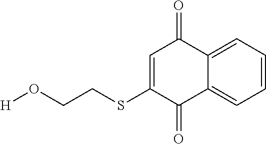 [SMILES: O=C1C=C(SCCO)C(=O)c2ccccc21] | 16 |
| NSC682996 | Quinoline-5,8-dione | 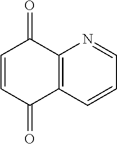 [SMILES: O=C1C=CC(=O)c2ncccc21] | 16 |
| NSC105808 | 6-amino-7-bromoquinoline-5,8-dione | 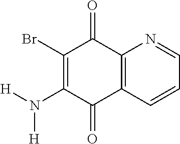 [SMILES: NC1=C(Br)C(=O)c2ncccc2C1=O] | 16 |
| NSC108361 | 4,5-dinitrophenanthrene-9,10-dione | 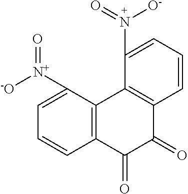 | 16 |
| NSC402968 | 2-nitro-10-nitrophenanthren-9-ol | 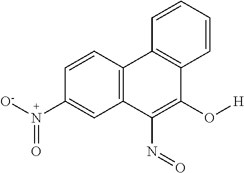 | 17 |
| NSC81050 | N-(5,8-dioxoquinolin-7-yl)acetamide | 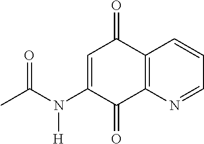 [SMILES: CC(=O)NC1=CC(=O)c2cccnc2C1=O] | 18 |
| NSC33530 | 2,7-dinitrophenanthrene-9,10-dione | 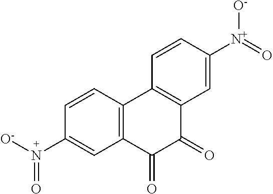 | 18 |
| NSC102364 | 2,7-dibromophenanthrene-9,10- dione | 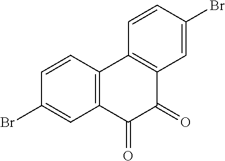 [SMILES: O=C1C(=O)c2cc(Br)ccc2-c2ccc(Br)cc21] | 19 |
| NSC5069 | Cacotheline | 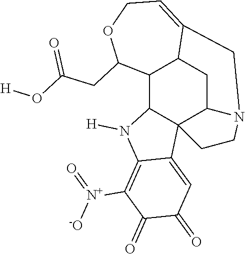 | 19 |
| NSC269980 | 3-(1,4-dioxonaphthalen-2- yl)sulfanylpropanoic acid | 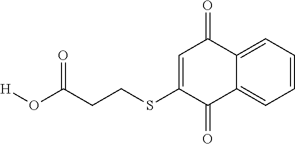 [SMILES: O=C(O)CCSC1=CC(=O)c2ccccc2C1=O] | 21 |
| NSC48648 | 2-ethylsulfanylnaphthalene-1,4- dione | 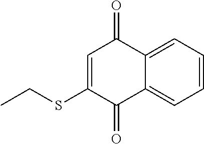 | 22 |
| NSC401103 | 2,3-dichloro-2,3- dihydronaphthalene-1,4-dione | 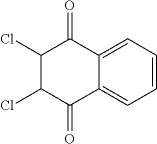 [SMILES: O=C1c2ccccc2C(=O)C(Cl)C1Cl] | 27 |
| NSC663286 | 7-chloro-6-(2- fluoroethylamino)quinoline-5,8-dione | 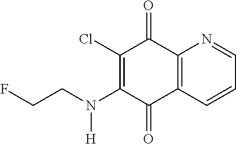 [SMILES: O=C1C(NCCF)=C(Cl)C(=O)c2ncccc21] | 28.7 |
| NSC105326 | 5-bromo-2-indol-3-ylidene-1H-indol- 3-one | 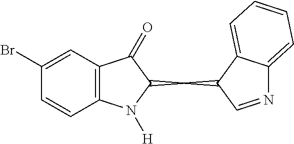 | 31 |
| NSC76886 | 6-aminoquinoline-5,8-dione | 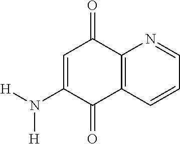 [SMILES: NC1=CC(=O)c2ncccc2C1=O] | 34 |
| NSC136107 | 10- (dibromomethylidene)phenanthren- 9-one | 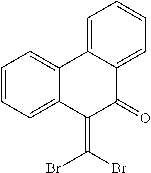 [SMILES: O=C1C(=C(Br)Br)c2ccccc2-c2ccccc21] | 35 |
| NSC115447 | 4,11-diaminonaphtho[2,3-f]isoindole- 1,3,5,10-tetrone | 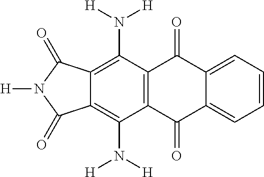 [SMILES: Nc1c2c(=O)[nH]c(=O)c2c(N)c2c(=O)c3ccccc3c(=O)c12] | 36 |
| Maybridge KM03663 | 5-methylsulfanyl-4-(4-methyl-1,3- thiazol-2-yl)thiophene-2- carbohydrazide 4-(4-methyl-1,3-thiazol-2-yl)-5- (methylthio)thiophene-2- carbohydrazide | 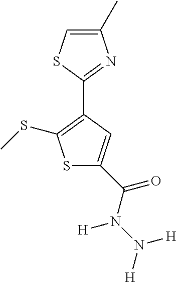 [SMILES: CSc1sc(C(=O)NN)cc1-c1nc(C)cs1] | 38 |
| NSC30982 | 2-(3-methyl-1,4-dioxonaphthalen-2- yl)sulfanylacetic acid | 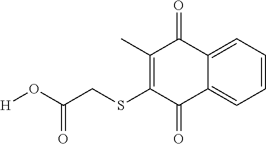 [SMILES: CC1=C(SCC(=O)O)C(=O)c2ccccc2C1=O] | 39 |
| NSC149109 | 2-buylsulfanylnaphhalene-1,4- dione | 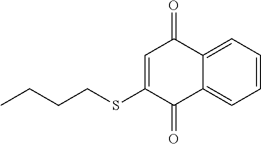 [SMILES: CCCCSC1=CC(=O)c2ccccc2C1=O] | 40 |
| Maybridge SP00172 | 2-{[5-(4-chlorophenyl)-2-methyl-3- furyl]carbonyl}-3-phenylacrylonitrile | 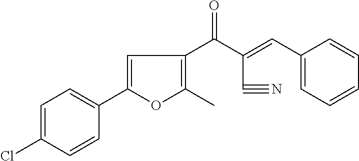 [SMILES: Cc1oc(-c2ccc(Cl)cc2)cc1C(=O)/C(C#N)=C/c1ccccc1] | 40 |
| Maybridge XAX00168 | 5-(5-nitro-2{[5-(trifluoromethyl)-2- pyridyl]thio}benzylidene)-2-thioxo- 1,3-thiozolan-4-one | 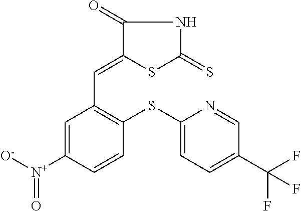 [SMILES: O=C1NC(=S)S/C1=C\c1cc([N+](=O)[O-])ccc1Sc1ccc(C(F)(F)F)cn1] | 46 |
| NSC90452 | 2-ethylsulfanyl-3- methylnaphthalene-1,4-dione | 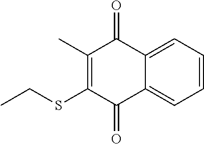 [SMILES: CCSC1=C(C)C(=O)c2ccccc2C1=O] | 51 |
| NSC672121 | 2-(2-hydroxyethylsulfanyl)-3- methylnaphthalene-1,4-dione | 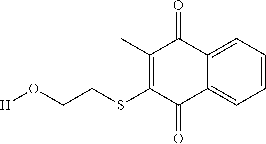 [SMILES: CC1=C(SCCO)C(=O)c2ccccc2C1=O] | 62 |
| NSC66166 | 2-methyl-3- methylsulfanylnaphthalene-1,4-dione | 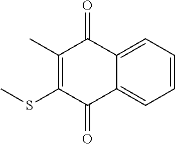 | 64 |
| Maybridge JFD03186 | (1,4-dioxonaphthalen-2-yl)-4- methylbenzoate | 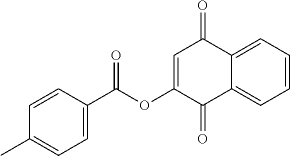 [SMILES: Cc1ccc(C(=O)OC2=CC(=O)c3ccccc3C2=O)cc1] | 64 |
| NSC93844 | 2-chloro-2,3-dihydronaphthalene- 1,4-dione | 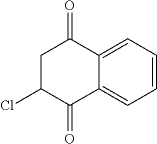 [SMILES: O=C1CC(Cl)C(=O)c2ccccc21] | 67.5 |
| NSC187762 | 6-[3- (dibutylamino)propylamino]quinoline- 5,8-dione | 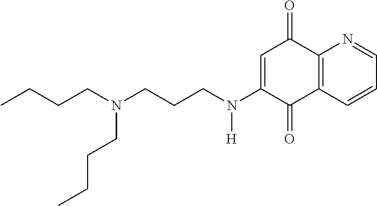 [SMILES: CCCCN(CCCC)CCCNC1=CC(=O)c2ncccc2C1=O] | 70 |
| NSC187761 | 6-(3-piperidin-1- ylpropylamino)quinoline-5,8-dione | 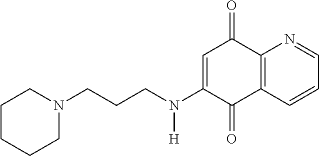 [SMILES: O=C1C(NCCCN2CCCCC2)=CC(=O)c2ncccc21] | 85 |
| NSC668394 | 7-[2-(3,5-dibromo-4- hydroxyphenyl)ethylamino]quinoline- 5,8-dione | 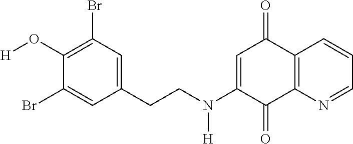 [SMILES: O=C1C=C(NCCc2cc(Br)c(O)c(Br)c2)C(=O)c2ncccc21] | 92 |
| NSC67209 | 2-methylsulfanylnaphthalene-1,4- dione | 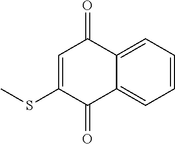 | 92.5 |
| NSC128981 | N-[3-(4-chlorophenyl)sulfanyl-1,4- dioxonaphthalen-2-yl]acetamide | 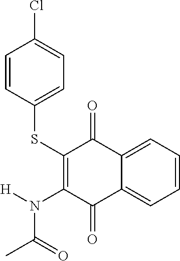 [SMILES: CC(=O)NC1=C(Sc2ccc(Cl)cc2)C(=O)c2ccccc2C1=O] | 94 |
| NSC66457 | 2-benzylsulfanyl-3- methylnaphthalene-1,4-dione | 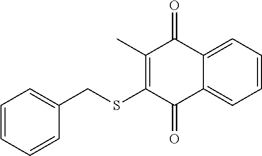 [SMILES: CC1=C(SCc2ccccc2)C(=O)c2ccccc2C1=O] | 95 |
| NSC130442 | N-(3-chloro-1,4-dioxonaphthalen-2- yl)-N-(4-fluorophenyl)acetamide | 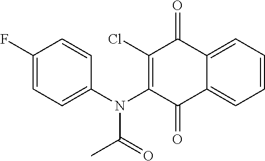 [SMILES: CC(=O)N(C1=C(Cl)C(=O)c2ccccc2C1=O)c1ccc(F)cc1] | 96 |

NSC numbers refer to compounds in the Developmental Therapeutics Program, National Cancer Institute (DTP/NCI) database. Maybridge identification numbers refer to compounds from Maybridge, part of Thermo Fisher Scientific. Some compounds have multiple NSC numbers. Some compounds reported a range of EC50 values. EC50 values were measured as described in the Experimental Details section.

As used herein “metastasize” means, in regard to a cancer or tumor, to spread from one organ or tissue of a patient to another non-adjacent organ or tissue of the patient. Preventing tumor metastasis means administering the agent or pharmaceutical composition thereof in a manner and amount sufficient to prevent clinically significant metastasis of a tumor. Inhibiting (i.e., treating) tumor metastasis means administering the agent or pharmaceutical composition thereof in a manner and amount sufficient to forestall the clinically significant metastasis of a tumor or to affect a clinically significant reduction in tumor metastasis (e.g., to reduce the number of metastases in an organ or tissue).

The present invention also provides a method of preventing or inhibiting tumor metastasis in a subject, the method comprising administering to the subject a therapeutically effective amount of an agent that covalently modifies at least one cysteine residue of an S100A4 protein, wherein the modification of at least one cysteine residue of S100A4 protein prevents or inhibits tumor metastasis in the subject.

S100A4 is a member of the S100 family of Ca²⁺-binding proteins and is directly involved in tumor metastasis. S100A4 modulates cellular motility by enhancing cell polarization, a direct consequence of S100A4's interaction with myosin-IIA, a major component of the motile machinery. S100A4 overexpression in epithelial tumor cells is associated with increased motility; whereas, reduction or loss of S100A4 expression correlates with decreased migration. S100A4 is a prognostic marker for a number of human cancers, including breast, esophageal-squamous cancers, non-small lung cancers, gastric cancers, malignant melanomas, prostate cancers, osteosarcoma, and bladder cancer.

The agent may covalently modify any cysteine residue in the S100A4 protein. Preferably, the agent covalently modifies cysteine residue 81 or cysteine residue 86.

The present invention further provides a method of preventing or inhibiting tumor metastasis in a subject, the method comprising administering to the subject a therapeutically effective amount of an agent that inhibits the interaction between S100A4 and myosin-IIA, wherein inhibition of the interaction between S100A4 and myosin-IIA prevents or inhibits tumor metastasis in the subject.

The agent may be any agent known in the art, for example, one containing at least one sulfur atom. In one example, the agent is 2,3-dihydrobenzo[g][1,4]benzodithiine-5,10-dione. In another example, the agent is 2,3-bis(2-hydroxyethylsulfanyl)naphthalene-1,4-dione. The agent may covalently modify the S100A4 protein by any method known in the art. For example, if the agent contains at least one sulfur atom, covalent modification of the S100A4 protein may comprise sulfhydryl arylation.

The agent or pharmaceutical composition can be administered by any method known in the art, including but not limited to, intravenously and orally.

The agent may be associated with a pharmaceutically-acceptable carrier, thereby comprising a pharmaceutical composition. The pharmaceutical composition may comprise the agent in a pharmaceutically acceptable carrier. Alternatively, the pharmaceutical composition may consist essentially of the agent in a pharmaceutically acceptable carrier. Yet alternatively, the pharmaceutical composition may consist of the agent in a pharmaceutically acceptable carrier.

The pharmaceutically-acceptable carrier must be compatible with the agent, and not deleterious to the subject. Examples of acceptable pharmaceutical carriers include carboxymethylcellulose, crystalline cellulose, glycerin, gum arabic, lactose, magnesium stearate, methyl cellulose, powders, saline, sodium alginate, sucrose, starch, talc, and water, among others. Formulations of the pharmaceutical composition may conveniently be presented in unit dosage and may be prepared by any method known in the pharmaceutical art. For example, the agent may be brought into association with a carrier or diluent, as a suspension or solution. Optionally, one or more accessory ingredients, such as buffers, flavoring agents, surface active ingredients, and the like, may also be added. The choice of carriers will depend on the method of administration. The pharmaceutical composition can be formulated to be administered by any method known in the art, including but not limited to, intravenously and orally. The pharmaceutical composition would be useful for administering the agent to a subject to prevent or inhibit tumor metastasis. The agent is provided in amounts effective to prevent or treat tumor metastasis in the subject. These amounts may be readily determined by one in the art. In one embodiment, the agent is the sole active pharmaceutical ingredient in the formulation or composition. In another embodiment, there may be a number of active pharmaceutical ingredients in the formulation or composition aside from the agent. In this embodiment, the other active pharmaceutical ingredients in the formulation or composition must be compatible with the agent.

An agent that prevents or inhibits the interaction between S100A4 and myosin-IIA may be determined by any method known in the art. For example, a fluorescence polarization assay that monitors the S100A4/myosin-IIA interaction using a fluorescein-tagged myosin-IIA peptide that comprises the minimal S100A4 binding site may be used as described in U.S. application Ser. No. 11/989,901, herein included in full.

The subject may be any mammal. For example, the subject is a human.

The tumor may be any tumor. For example, the tumor may be a tumor of the breast, esophagus, pulmonary system, digestive tract, skin, prostate, bone, bladder, pancreas, ovary, kidney, brain, liver, head or neck.

The present invention provides the compound or agent as described in any of the method claims for use in preventing or inhibiting tumor metastasis in a subject.

### Experimental Details

### 1. Background

### S100A4 has a Causative Role in Cancer Metastasis

S100A4 is a member of the S100 family of Ca²⁺-binding proteins and is directly involved in tumor metastasis. Evidence from animal models and studies of human breast cancer indicate that S100A4 is not simply a marker for metastatic disease, but rather has a direct role in mediating this process. Of particular relevance to the role of S100A4 in promoting a metastatic phenotype are studies demonstrating that S100A4 overexpression in epithelial tumor cells is associated with increased motility; whereas, reduction or loss of S100A4 expression correlates with decreased migration (2-6). Thus, potent and specific S100A4 inhibitors will target a central element of the metastatic cascade (i.e., motility) and may represent new therapeutics for the treatment of metastatic disease. The contribution of S100A4 to metastatic progression has been most widely examined in breast cancer (5, 7-13); however, S100A4 is a prognostic marker for a number of human cancers, including esophageal-squamous cancers (14), non-small lung cancers (15), gastric cancers (16), malignant melanomas (17) and prostate cancers (18, 19). Moreover, S100A4 overexpression enhances malignant potential in animal models of osteosarcoma, prostate and bladder cancer. The universality of S100A4 expression suggests that S100A4 contributes to metastasis and disease progression in a variety of cancers, and highlights the potential use of S100A4 inhibitors in the treatment of many cancer types.

### S100A4, the Actomyosin Cytoskeleton and Motility

Studies demonstrate that S100A4 preferentially binds to the C-terminal end of the coiled-coil of the myosin-IIA heavy chain in a Ca²⁺-dependent manner, and promotes the monomeric, unassembled state of myosin-IIA (20, 21). Investigations also show that S100A4 modulates cellular motility by enhancing cell polarization, and that this is a direct consequence of S100A4's interaction with myosin-IIA (22). These findings establish S100A4 as a critical regulator of myosin-II function and motility, which is a central element of the metastatic cascade.

S100A4 Interacts with Protein Targets Via a Ca²⁺-Dependent Conformational Rearrangement

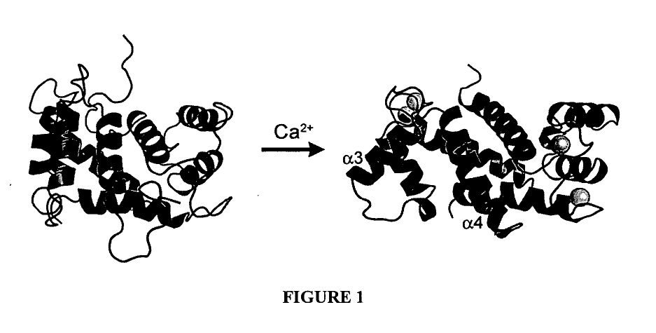

Each S100A4 monomer contains two Ca²⁺-binding loops; a C-terminal ‘typical’ EF-hand comprised of 12 residues and an N-terminal pseudo EF-hand consisting of 14 residues. Structural studies demonstrate that S100A4 is a symmetric, antiparallel homodimer, in which the N- and C-terminal helices (helices 1 and 4) from each subunit interact to form a stable four helix bundle that serves as the dimer interface (23) (FIG. 1). Calcium binding to the C-terminal typical EF-hand significantly alters the angle between helices 3 and 4, which flank the C-terminal Ca²⁺-binding loop, and exposes a hydrophobic cleft that constitutes the binding surface for target proteins (21).

The present invention identifies compounds that bind S100A4 and disrupt the interaction of S100A4 with its protein target myosin-HA, a major component of the motile machinery. The interaction with myosin-HA provides a direct link between S100A4, the cytoskeleton, and tumor cell motility/invasion. These efforts also show that S100A4 is a “druggable” target.

### 2. Results and Discussion

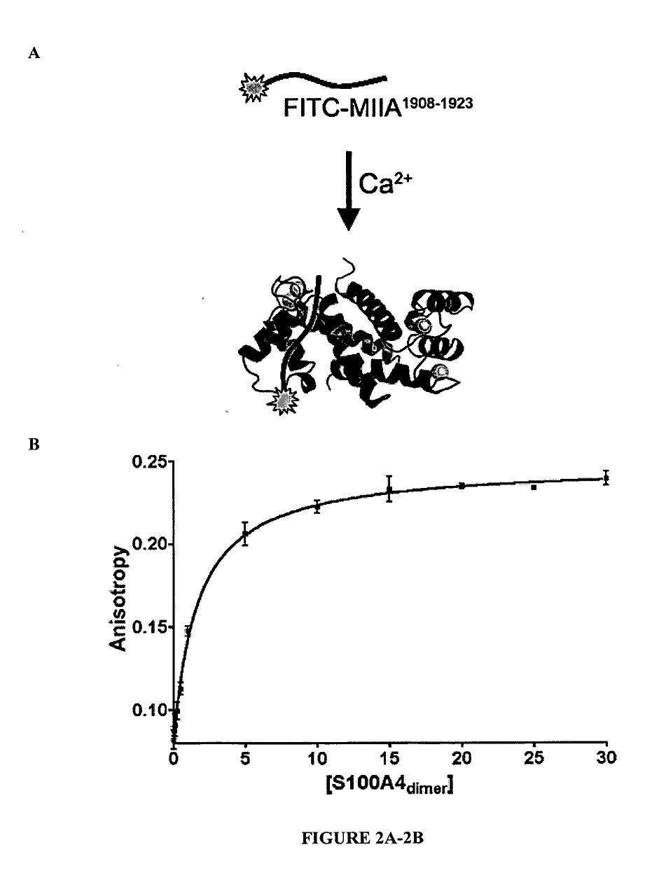

A fluorescence polarization assay was developed that monitors the S100A4/myosin-IIA interaction using a fluorescein-tagged myosin-IIA peptide that comprises the minimal S100A4 binding site (FITC-MIIA¹⁹⁰⁸⁻¹⁹²³) (21, 24). FITC-MIIA¹⁹⁰⁸⁻¹⁹²³ exhibits Ca²⁺-dependent binding to S100A4 with a K_{d} of 1.7±0.2 μM (FIG. 2). In collaboration with the Rapid Access to NCI Discovery Resources (R•A•N•D) program, the fluorescence polarization assay was used to screen the LOPAC (1280 compounds) and Maybridge (14,320 compounds) chemical libraries to identify compounds that disrupt the S100A4/myosin-IIA interaction. From the LOPAC and Maybridge screens, NSC 95397 (EC50=7.6 μM) and SP00172, XAX00168 and KM03663 (EC50 values 38-46 μM) were identified, respectively, as inhibitors of the S100A4/myosin-IIA interaction. In addition, in a structure-activity relationship (SAR) study of 103 compounds related to NSC 95397, it was demonstrated that the phenanthroquinone chemotype is a more potent inhibitor of S100A4 with nine compounds exhibiting EC50 values in the 0.5-4 μM range.

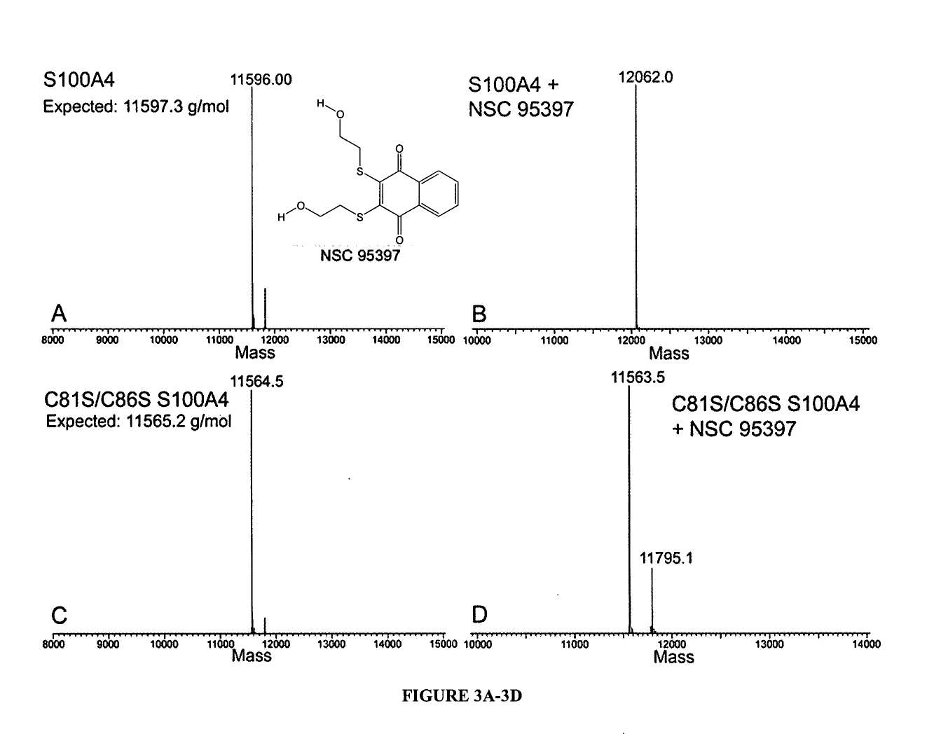

Since NSC 95397 has been characterized previously as an irreversible inhibitor of other signaling molecules via sulfhydryl arylation of active site cysteines, it was examined whether NSC 95397 can covalently modify S100A4. Following treatment with NSC 95397, the mass of S100A4 indicates modification of each monomer at two residues (FIGS. 3A and B), and is consistent with the loss of one hydroxyethylsulfanyl moiety per NSC 95397 molecule. Substitution of Cys81 and Cys86 with serine prevented modification by NSC 95397 (FIGS. 3C and D), suggesting that cysteines 81 and 86, which reside in the target binding cleft, are the primary sites of modification. Biochemical studies indicate that C3R/C86S S100A4 binds FITC-MIIA¹⁹⁰⁸⁻¹⁹²³ with wild-type activity (K_{d} of 1.2±0.1 μM); however, myosin-IIA binding to C3R/C81S/C86S S100A4 was undetectable. These observations suggest that Cys81 is critical for the interaction of S100A4 with myosin-IIA, and demonstrate that these small molecule screens are revealing important information about the S100A4/myosin-IIA interaction.

NSC 95397, the three Maybridge compounds and seven compounds from the SAR study were obtained for additional biological testing. The loss of S100A4 expression due to genetic deletion does not effect cell proliferation, thus compounds inhibiting S100A4 activity should not affect overall cell growth. However, all compounds were tested in a cell proliferation assay to identify cytotoxic compounds and determine compound concentrations that can be used to examine effects on motility and invasion. The MTT assay revealed that some of the phenanthroquinones tested are toxic to human breast cancer cells at concentrations required to disrupt S100A4 function (Table 2). However, characterization of all compounds in structural and biochemical studies can impart essential information on the mechanism of S100A4 inhibition and aid in the development of future inhibitors. Importantly, NSC 5069 (EC50=19 μM), which was identified in the SAR study, did not exhibit toxicity at any concentration tested. In addition, Maybridge compound XAX 00168 (EC50 value=46 μM) has a GI50 of 190 μM, thus allowing for biological evaluation. A full list of compounds identified in the screens is in Table 1.

TABLE 2
Summary of compounds identified in the LOPAC and Maybridge screens and SAR study.

| | | EC50 (♦M) | |
|---|---|---|---|
| | Chemical Name | FP Assay | MTT Assay |
| Compound | | | |
| NSC 95397 | 2,3-Bis[(2-hydroxyethyl)thio]-1,4-naphthoquinone | 7.6 | 100% death @ 2.5 μM |
| NSC 55480 | 2,3-Dihydronaphtho[2,3-b][1,4]dithiine-5,10-dione | 2.2 | 100% death @ 2.5 μM |
| NSC 5425 | 2-nitrophenanthrene-9,10-dione | 0.5 | 100% death @ 10 μM |
| NSC 102363 | 2-chlorophenanthrene-9,10-dione | 1.8 | 100% death @ 10 μM |
| NSC 77642 | 2-Amino-phenanthrene-9,10-dione | 1.6 | not available for testing |
| NSC 23180 | 2-Nitro-9,10-phenanthrenedione | 2 | not available for testing |
| NSC 400689 | 3-acetylphenanthrene-9,10-dione | 2.2 | 100% death @ 2.5 μM |
| NSC 10204 | 4-nitrophenanthrene-9,10-dione | 2.5 | 80% death at 100 μM |
| NSC 6339 | 10,10-dichlorophenanthren-9-one | 2.5 | 100% death @ 2.5 μM |
| NSC 7389 | phenanthrene-9,10-dione | 3.8 | 100% death @ 25 μM |
| Singletons | | | |
| NSC 5069 | Cacotheline | 19 | no toxicity detected |
| Maybridge | | | |
| Compounds | | | |
| KM 03663 | 4-(4-methyl-1,3-thiazol-2-yl)-5-(methylthio)thiophene-2-carbohydrazide | 38 | 100% death at 150 μM |
| SP 00172 | 2-{[5-(4-chlorophenyl)-2-methyl-3-furyl]carbonyl}-3-phenylacrylonitrile | 40 | 100% death at 50 μM |
| XAX 00168 | 5-(5-nitro-2-{[5-(trifluoromethyl)-2-pyridyl]thio}benzylidene)- | 46 | IC50 = 190.3 μM |
| | 2-thioxo-1,3-thiazolan-4-one | | |

Hits were identified from fluorescence polarization assays using four compound concentrations and confirmed in assays using sixteen compound concentrations. EC50 values were determined from sixteen concentration titrations. Growth inhibition of MDA-MB-231 cells, which express high levels of S100A4, was examined in a 96-well plate MTT assay at eight drug concentrations after treatment for 24 hrs.

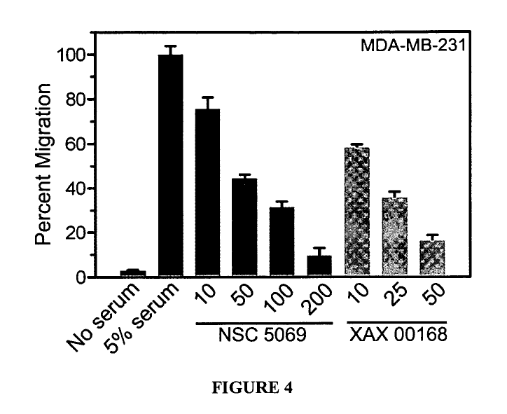

As proof-of-principle that S100A4 inhibitors affect the motile and invasive capabilities of carcinoma cells, cell migration assays examining the ability of NSC 5069 and XAX 00168 to inhibit chemotaxis of MDA-MB-231 cells towards serum in a Boyden chamber assay were initiated (FIG. 4). Both NSC 5069 and XAX 00168 inhibit serum-stimulated migration to varying extents with EC50 values of approximately 40 and 20 μM for NSC 5069 and XAX 00168, respectively.

### REFERENCES

1. Stetler-Stevenson, W. G., Aznavoorian, S., and Liotta, L. A. (1993) Tumor cell interactions with the extracellular matrix during invasion and metastasis., Annu Rev Cell Biol 9, 541-573.
2. Takenaga, K., Nakamura, Y., Endo, H., and Sakiyama, S. (1994) Involvement of S100-related calcium-binding protein pEL98 (or mts1) in cell motility and tumor cell invasion, Jpn J Cancer Res 85, 831-839.
3. Chen, P. S., Wang, M. Y., Wu, S, N., Su, J. L., Hong, C. C., Chuang, S. E., Chen, M. W., Hua, K. T., Wu, Y. L., Cha, S. T., Babu, M. S., Chen, C. N., Lee, P. H., Chang, K. J., and Kuo, M. L. (2007) CTGF enhances the motility of breast cancer cells via an integrin-alphavbeta3-ERK1/2-dependent S100A4-upregulated pathway, J Cell Sci 120, 2053-2065.
4. Bjornland, K., Winberg, J. O., Odegaard, O. T., Hovig, E., Loennechen, T., Aasen, A. O., Fodstad, O., and Maelandsmo, G. M. (1999) S100A4 involvement in metastasis: deregulation of matrix metalloproteinases and tissue inhibitors of matrix metalloproteinases in osteosarcoma cells transfected with an anti-S100A4 ribozyme, Cancer Res 59, 4702-4708.
5. Takenaga, K., Nakamura, Y., and Sakiyama, S. (1997) Expression of antisense RNA to S100A4 gene encoding an S100-related calcium-binding protein suppresses metastatic potential of high-metastatic Lewis lung carcinoma cells, Oncogene 14, 331-337.
6. Stein, U., Arlt, F., Walther, W., Smith, J., Waldman, T., Harris, E. D., Mertins, S. D., Heizmann, C. W., Allard, D., Birchmeier, W., Schlag, P. M., and Shoemaker, R. H. (2006) The metastasis-associated gene S100A4 is a novel target of beta-catenin/T-cell factor signaling in colon cancer, Gastroenterology 131, 1486-1500.
7. Davies, B. R., Davies, M. P., Gibbs, F. E., Barraclough, R., and Rudland, P. S. (1993) Induction of the metastatic phenotype by transfection of a benign rat mammary epithelial cell line with the gene for p9Ka, a rat calcium-binding protein, but not with the oncogene EJ-ras-1., Oncogene 8, 999-1008.
8. Grigorian, M., Ambartsumian, N., Lykkesfeldt, A. E., Bastholm, L., Elling, F., Georgiev, G., and Lukanidin, E. (1996) Effect of mts1 (S100A4) expression on the progression of human breast cancer cells., Int J Cancer 67, 831-841.
9. Maelandsmo, G. M., Hovig, E., Skrede, M., Engebraaten, O., Florenes, V. A., Myklebost, O., Grigorian, M., Lukanidin, E., Scanlon, K. J., and Fodstad, O. (1996) Reversal of the in vivo metastatic phenotype of human tumor cells by an anti-CAPL (mts1) ribozyme., Cancer Res 56, 5490-5498.
10. Davies, M. P., Rudland, P. S., Robertson, L., Parry, E. W., Jolicoeur, P., and Barraclough, R. (1996) Expression of the calcium-binding protein S100A4 (p9Ka) in MMTV-neu transgenic mice induces metastasis of mammary tumours., Oncogene 13, 1631-1637.
11. Xue, C., Plieth, D., Venkov, C., Xu, C., and Neilson, E. G. (2003) The gatekeeper effect of epithelial-mesenchymal transition regulates the frequency of breast cancer metastasis, Cancer Res 63, 3386-3394.
12. Lee, W. Y., Su, W. C., Lin, P. W., Guo, H. R., Chang, T. W., and Chen, H. H. (2004) Expression of S100A4 and Met: potential predictors for metastasis and survival in early-stage breast cancer, Oncology 66, 429-438.
13. de Silva Rudland, S., Martin, L., Roshanlall, C., Winstanley, J., Leinster, S., Platt-Higgins, A., Carroll, J., West, C., Barraclough, R., and Rudland, P. (2006) Association of S100A4 and osteopontin with specific prognostic factors and survival of patients with minimally invasive breast cancer, Clin Cancer Res 12, 1192-1200.
14. Ninomiya, I., Ohta, T., Fushida, S., Endo, Y., Hashimoto, T., Yagi, M., Fujimura, T., Nishimura, G., Tani, T., Shimizu, K., Yonemura, Y., Heizmann, C. W., Schafer, B. W., Sasaki, T., and Miwa, K. (2001) Increased expression of S100A4 and its prognostic significance in esophageal squamous cell carcinoma, Int J Oncol 18, 715-720.
15. Kimura, K., Endo, Y., Yonemura, Y., Heizmann, C. W., Schafer, B. W., Watanabe, Y., and Sasaki, T. (2000) Clinical significance of S100A4 and E-cadherin-related adhesion molecules in non-small cell lung cancer, Int J Oncol 16, 1125-1131.
16. Yonemura, Y., Endou, Y., Kimura, K., Fushida, S., Bandou, E., Taniguchi, K., Kinoshita, K., Ninomiya, I., Sugiyama, K., Heizmann, C. W., Schafer, B. W., and Sasaki, T. (2000) Inverse expression of S100A4 and E-cadherin is associated with metastatic potential in gastric cancer, Clin Cancer Res 6, 4234-4242.
17. Andersen, K., Nesland, J. M., Holm, R., Florenes, V. A., Fodstad, O., and Maelandsmo, G. M. (2004) Expression of S100A4 combined with reduced E-cadherin expression predicts patient outcome in malignant melanoma, Mod Pathol 17, 990-997.
18. Gupta, S., Hussain, T., MacLennan, G. T., Fu, P., Patel, J., and Mukhtar, H. (2003) Differential expression of S100A2 and S100A4 during progression of human prostate adenocarcinoma, J Clin Oncol 21, 106-112.
19. Saleem, M., Adhami, V. M., Ahmad, N., Gupta, S., and Mukhtar, H. (2005) Prognostic significance of metastasis-associated protein S100A4 (Mts1) in prostate cancer progression and chemoprevention regimens in an autochthonous mouse model, Clin Cancer Res 11, 147-153.
20. Li, Z.-H., Spektor, A., Varlamova, 0., and Bresnick, A. R. (2003) Mts1 regulates the assembly of nonmuscle myosin-IIA., Biochemistry 42, 14258-14266.
21. Malashkevich, V. N., Varney, K. M., Garrett, S. C., Wilder, P. T., Knight, D., Charpentier, T. H., Ramagopal, U. A., Almo, S. C., Weber, D. J., and Bresnick, A. R. (2008) Structure of Ca2+-bound S100A4 and its interaction with peptides derived from nonmuscle myosin-IIA, Biochemistry 47, 5111-5126.
22. Li, Z. H., and Bresnick, A. R. (2006) The S100A4 metastasis factor regulates cellular motility via a direct interaction with myosin-IIA, Cancer Research 66, 5173-5180.
23. Vallely, K. M., Rustandi, R. R., Ellis, K. C., Varlamova, O., Bresnick, A. R., and Weber, D. J. (2002) Solution structure of human mts1 (S100A4) as determined by NMR spectroscopy., Biochemistry 41, 12670-12680.
24. Garrett, S. C., Hodgson, L., Rybin, A., Toutchkine, A., Hahn, K. M., Lawrence, D. S., and Bresnick, A. R. (2008) A biosensor of S100A4 metastasis factor activation: inhibitor screening and cellular activation dynamics, Biochemistry 47, 986-996.

1. A method of preventing or inhibiting tumor metastasis in a subject, the method comprising administering to the subject a therapeutically effective amount of a compound selected from the group consisting of 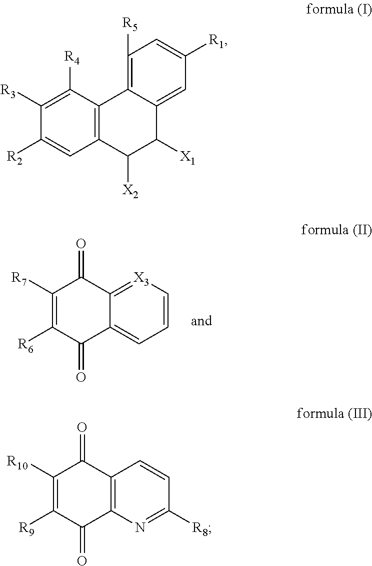
wherein
X1 is ( )═O or ( )—OH;
X2 is selected from the group consisting of 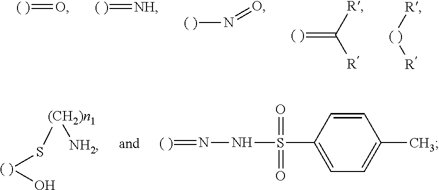
wherein R′ is a halogen and n₁ is an integer 0-3;
X3 is CH or N;
R1 is selected from the group consisting of H, Br, Cl, F, I, At, and 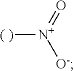
R2, R3 and R4 are independently selected from the group consisting of 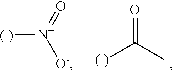
H, NH₂, Cl, Br, F, I, and At;
R5 is H or 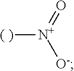
R6 is selected from the group consisting of Cl, F, Br, I, At, NH₂, H, 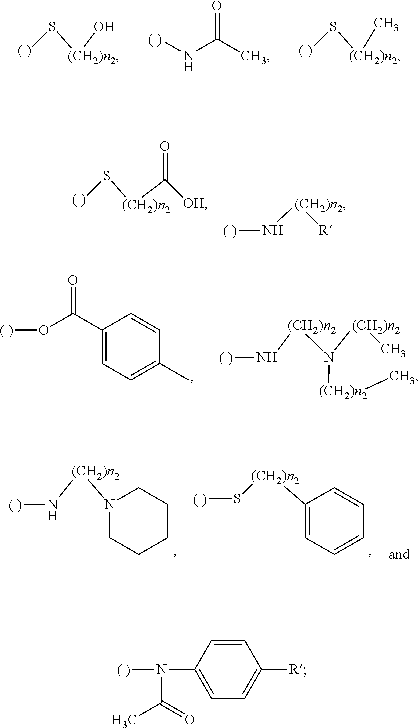
wherein R′ is a halogen and each n₂ is independently an integer 0-5;
R7 is selected from the group consisting of H, Cl, Br, F, I, At, CH₃, 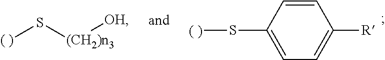
wherein R′ is a halogen and n₃ is an integer 0-5;
R8 is CH₃ or H;
R9 is selected from the group consisting of H, NH₂, 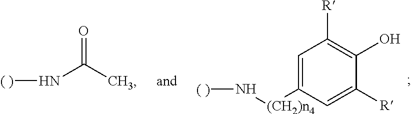
wherein R′ is a halogen and n₄ is an integer 0-5; and
R10 is H or 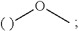
wherein R′ is a halogen, n₁ is an integer 0-3, and n₂ is an integer 0-5;
wherein ( ) is the point of attachment of the X or R group to the ring structure;
or a pharmaceutically acceptable salt thereof.

2. The method of claim 1, wherein the compound is the compound of formula (I).

3. The method of claim 1, wherein formula (I) is selected from the group consisting of 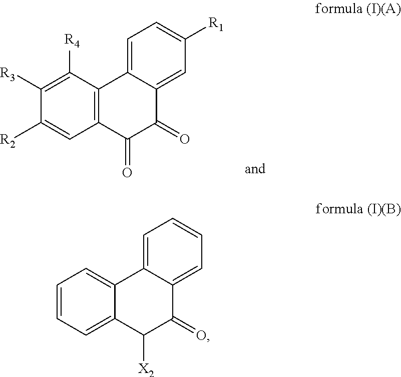
wherein
X2 is selected from the group consisting of 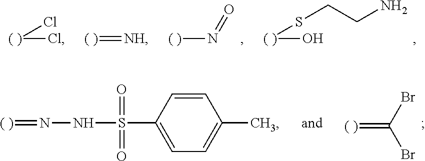
R1 is selected from the group consisting of H, Br, Cl, and 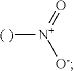
R2 is selected from the group consisting of H, NH₂, Cl, Br and 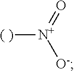
R3 is H or 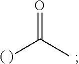
and
R4 is H or

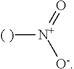

4. The method of claim 1, wherein formula (II) is selected from the group consisting of 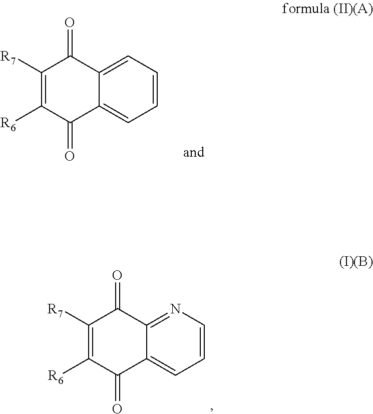
wherein
R6 is selected from the group consisting of Cl, F, Br, H, 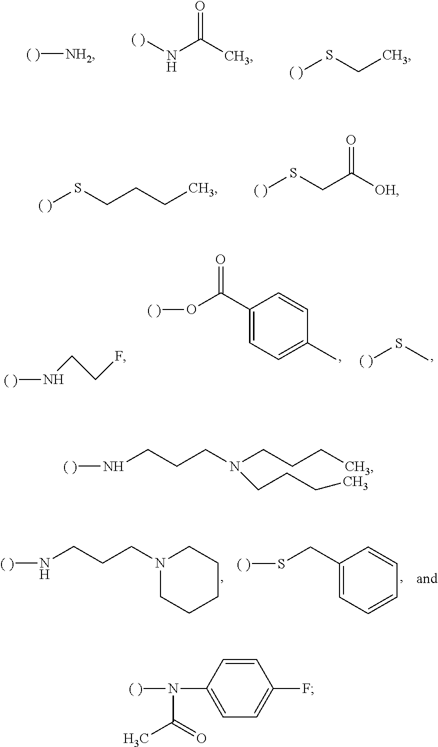
and
R7 is selected from the group consisting of Cl, Br, H, CH₃,

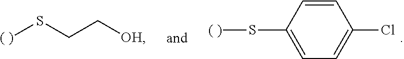

5. The method of claim 1, wherein the compound is the compound of formula (III).

6. The method of claim 1, wherein the compound is a compound of formula (III), wherein
R9 is selected from the group consisting of H, NH₂,

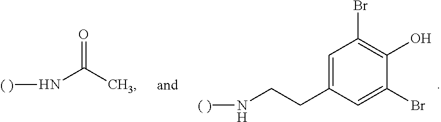

7. A method of preventing or inhibiting tumor metastasis in a subject, the method comprising administering a therapeutically effective amount of a compound selected from the group consisting of:

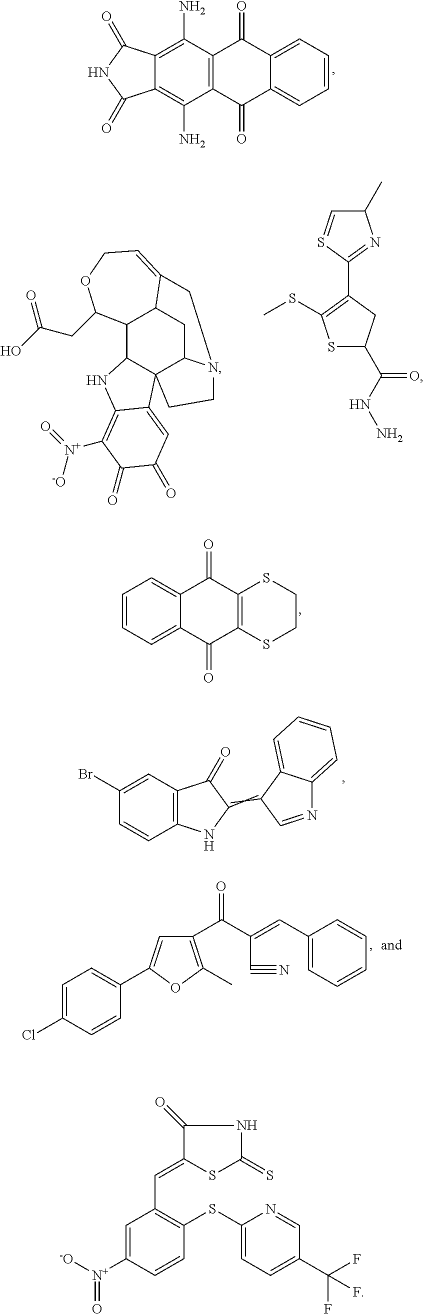

8. The method of claim 1, wherein the compound comprises 2,3-dihydrobenzo[g][1,4]benzodithiine-5,10-dione, 2,3-bis(2-hydroxyethylsulfanyl)naphthalene-1,4-dione, 2-(2-hydroxyethylsulfanyl)naphthalene-1,4-dione, 3-(1,4-dioxonaphthalen-2-yl)sulfanylpropanoic acid, 2-ethylsulfanylnaphthalene-1,4-dione, 4,11-diaminonaphtho[2,3-f]isoindole-1,3,5,10-tetrone, 2-(3-methyl-1,4-dioxonaphthalen-2-yl)sulfanylacetic acid, 2-butylsulfanylnaphthalene-1,4-dione, 2-ethylsulfanyl-3-methylnaphthalene-1,4-dione, 2-(2-hydroxyethylsulfanyl)-3-methylnaphthalene-1,4-dione, 2-methyl-3-methylsulfanylnaphthalene-1,4-dione, (1,4-dioxonaphthalen-2-yl) 4-methylbenzoate, N-[3-(4-chlorophenyl)sulfanyl-1,4-dioxonaphthalen-2-yl]acetamide, 2-benzylsulfanyl-3-methylnaphthalene-1,4-dione, N-(3-chloro-1,4-dioxonaphthalen-2-yl)-N-(4-fluorophenyl)acetamide, 2-methylquinoline-5,8-dione, N-(7-chloro-5,8-dioxoquinolin-6-yl)acetamide, 6-amino-7-chloroquinoline-5,8-dione, 7-amino-6-methoxyquinoline-5,8-dione, 6,7-dichloroquinoline-5,8-dione, quinoline-5,8-dione, 6-amino-7-bromoquinoline-5,8-dione, N-(5,8-dioxoquinolin-7-yl)acetamide, 2,3-dichloro-2,3-dihydronaphthalene-1,4-dione, 7-chloro-6-(2-fluoroethylamino)quinoline-5,8-dione, 6-aminoquinoline-5,8-dione, 2-chloro-2,3-dihydronaphthalene-1,4-dione, 6-[3-(dibutylamino)propylamino]quinoline-5,8-dione, 6-(3-piperidin-1-ylpropylamino)quinoline-5,8-dione, 2-methylsulfanylnaphthalene-1,4-dione, 2-nitrophenanthrene-9,10-dione, 2-chlorophenanthrene-9,10-dione, 2-aminophenanthrene-9,10-dione, 3-acetylphenanthrene-9,10-dione, 4-nitrophenanthrene-9,10-dione, 10,10-dichlorophenanthren-9-one, phenanthrene-9,10-dione, 10-(2-aminoethylsulfanyl)-10-hydroxyphenanthren-9-one hydrochloride, 10-iminophenanthren-9-one, 2,7-dichlorophenanthrene-9,10-dione, 2,7-dibromo-4-nitrophenanthrene-9,10-dione, 4-methyl-N—[(Z)-(10-oxophenanthren-9-ylidene)amino]benzenesulfonamide, 10-nitrosophenanthren-9-ol, 4,5-dinitrophenanthrene-9,10-dione, 2-nitro-10-nitrosophenanthren-9-ol, 2,7-dinitrophenanthrene-9,10-dione, 2,7-dibromophenanthrene-9,10-dione, 10-(dibromomethylidene)phenanthren-9-one, Cacotheline, 5-bromo-2-indol-3-ylidene-1H-indol-3-one, 7-[2-(3,5-dibromo-4-hydroxyphenyl)ethylamino]quinoline-5,8-dione, 5-methylsulfanyl-4-(4-methyl-1,3-thiazol-2-yl)thiophene-2-carbohydrazide, 2-{[5-(4-chlorophenyl)-2-methyl-3-furyl]carbonyl}-3-phenylacrylonitrile, or 5-(5-nitro-2{[5-(trifluoromethyl)-2-pyridyl]thio}benzylidene)-2-thioxo-1,3-thiozolan-4-one.

9. The method of claim 8, the method comprising administering a therapeutically effective amount of 2,3-dihydrobenzo[g][1,4]benzodithiine-5,10-dione.

10-37. (canceled)

38. The method of claim 8, the method comprising administering a therapeutically effective amount of 2-nitrophenanthrene-9,10-dione.

39. The method of claim 8, the method comprising administering a therapeutically effective amount of 2-chlorophenanthrene-9,10-dione.

40. The method of claim 8, the method comprising administering a therapeutically effective amount of 2-aminophenanthrene-9,10-dione.

41. The method of claim 8, the method comprising administering a therapeutically effective amount of 3-acetylphenanthrene-9,10-dione.

42. The method of claim 8, the method comprising administering a therapeutically effective amount of 4-nitrophenanthrene-9,10-dione.

43. The method of claim 8, the method comprising administering a therapeutically effective amount of 10,10-dichlorophenanthren-9-one.

44. The method of claim 8, the method comprising administering a therapeutically effective amount of phenanthrene-9,10-dione.

45-61. (canceled)

62. A method of preventing or inhibiting tumor metastasis in a subject, the method comprising administering to the subject a therapeutically effective amount of an agent that covalently modifies at least one cysteine residue of S100A4 protein, wherein the modification of at least one cysteine residue of S100A4 protein prevents or inhibits tumor metastasis in the subject.

63-67. (canceled)

68. A method of preventing or inhibiting tumor metastasis in a subject, the method comprising administering to the subject a therapeutically effective amount of an agent that inhibits the interaction between S100A4 and myosin-IIA, wherein inhibition of the interaction between S100A4 and myosin-IIA prevents or inhibits tumor metastasis in the subject.

69. (canceled)

70. The method of claim 1, wherein the subject is a human.

71. The method of claim 1, wherein the tumor comprises a tumor in the breast, esophagus, pulmonary system, digestive tract, skin, prostate, bone, bladder, pancreas, ovary, kidney, brain, liver, head or neck.

72-75. (canceled)
